(19) United States
(12) Patent Application Publication
(10) Pub. No.: US 2016/0030008 A1
(43) Pub. Date: Feb. 4, 2016

(54) SYSTEM AND METHOD FOR REGISTERING ULTRASOUND INFORMATION TO AN X-RAY IMAGE

(71) Applicant: General Electric Company, Schenectady, NY (US)

(72) Inventor: Olivier Gerard, Horten (NO)

(21) Appl. No.: 14/446,498

(22) Filed: Jul. 30, 2014

(51) Int. Cl. A61B 8/08; A61B 8/00

(52) U.S. Cl. CPC A61B 8/5261; A61B 8/463; A61B 8/54

(57) ABSTRACT

A system and a method of medical imaging includes registering an ultrasound image to a non-ultrasound image according to a first transformation. The system and method includes registering the non-ultrasound image to the x-ray image according to a second transformation. The system and method includes registering the ultrasound image to the x-ray image based on the first transformation and the second transformation and co-displaying ultrasound information registered to the x-ray image. The ultrasound information is based on the ultrasound data.

### FIELD OF THE INVENTION

This disclosure relates generally to an ultrasound imaging system and method of registering ultrasound information to an x-ray image.

### BACKGROUND OF THE INVENTION

Different imaging modalities have different strengths and weaknesses for imaging various anatomical structures. For example, CT images, which reconstruct images based on x-ray attenuation data, are relatively quick to acquire and accurately depict the anatomical structure being imaged. CT images are excellent for imaging hard or bony tissue, but they are less well-suited for imaging soft tissue. MRI images, on the other hand, generate images based on the proton density of various tissues. MRI images take longer to acquire than CT images, but they are more well-suited for imaging soft tissue. Neither CT nor MRI are ideal as real-time imaging modalities. CT is limited for reasons related to x-ray dose, while MRI is impractical for any procedures that would require the use of ferrous instruments or implantable devices due to the high magnetic field generated by the magnet. Neither CT nor MRI imaging is ideal for wide-spread use in real-time procedures as the imaging systems are large and expensive, and they include a tube-shaped bore where the patient is positioned that makes access to the patient difficult or impractical. Additionally, MRI images are relatively slow to acquire which makes the modality less useful for real-time procedures.

If real-time feedback is required, modalities such as x-ray fluoroscopy or ultrasound are better choices for most applications. X-ray fluoroscopy uses low-dose x-rays to generate a real-time x-ray image. X-ray fluoroscopy is commonly used during interventional procedures to provide a surgeon with real-time feedback during the procedure. Like CT, x-ray fluoroscopy is an excellent choice for visualizing hard tissue, such as bones, and/or visualizing interventional devices within a patient. X-ray fluoroscopy is not the most diagnostically useful modality for imaging soft tissue. Ultrasound, on the other hand, is well-suited for imaging soft tissue. Ultrasound, however, does not always provide clear images of interventional devices, which are typically made of metal and tend to be small in diameter. Ultrasound images do not always provide an accurate representation of the position of interventional devices in a patient's body.

Combining information from different imaging modalities is useful during intervention procedures. For example, during interventional procedures, including many common cardiac procedures, it is desirable to combine a real-time, or live, ultrasound image with an x-ray fluoroscopy image. The ultrasound image provides real-time information about soft tissue while the x-ray fluoroscopy image clearly shows hard structures, such as the interventional device and bones within the patient. X-ray fluoroscopy is not well-suited for visualizing soft tissue.

Conventional techniques exist for registering ultrasound images with x-ray fluoroscopy images. Most of these techniques require an external tracking system, such as an optical tracking system or an electromagnetic tracking system. Using an external tracking system is undesirable for several reasons. The external tracking system adds cost and complexity to the system. Additionally, in order to track an interventional device, it is necessary to mount a tracking device on the interventional device. Mounting a tracking device on the interventional device increases the cost of the interventional device. This may be particularly problematic for disposable or single use interventional devices. Including a tracking device results in an interventional device that is at least one of heavier, bulkier, and more expensive than a conventional interventional device. Additionally, some types of interventional devices may not currently be available with an integrated tracking device.

For these and other reasons an improved ultrasound imaging system and method for registering ultrasound information to x-ray images is desired.

### BRIEF DESCRIPTION OF THE INVENTION

The above-mentioned shortcomings, disadvantages and problems are addressed herein which will be understood by reading and understanding the following specification.

In an embodiment, a method of medical imaging includes accessing ultrasound data, generating an ultrasound image based on the ultrasound data, and accessing a non-ultrasound image and an x-ray image. The method includes registering the ultrasound image to the non-ultrasound image according to a first transformation, registering the non-ultrasound image to the x-ray image according to a second transformation, and registering the ultrasound image to the x-ray image based on the first transformation and the second transformation. The method includes co-displaying ultrasound information registered to the x-ray image, where the ultrasound information is based on the ultrasound data.

In an embodiment, an ultrasound imaging system includes a probe, a display device, and a processor in electronic communication with the probe and the display device. The processor is configured to control the probe to acquire ultrasound data, access a non-ultrasound image, and access an x-ray image. The processor is configured to calculate a first transformation to register the x-ray image to the non-ultrasound image, calculate a second transformation to register the ultrasound image to the non-ultrasound image, and calculate a third transformation to register the ultrasound image to the x-ray image based on both the first transformation and the second transformation. The processor is configured to co-display ultrasound information registered to the x-ray image on the display device, wherein the ultrasound information is based on the ultrasound data.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of an ultrasound imaging system in accordance with an embodiment;

FIG. 2 is a schematic diagram of a system in accordance with an embodiment;

FIG. 3 is a flow chart of a method in accordance with an embodiment;

FIG. 4 is a schematic representation of a screenshot in accordance with an embodiment;

FIG. 5 is a schematic representation of a screenshot in accordance with an embodiment;

FIG. 6 is a schematic representation of a screenshot in accordance with an embodiment; and

FIG. 7 is a schematic representation of a screenshot according to an exemplary embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

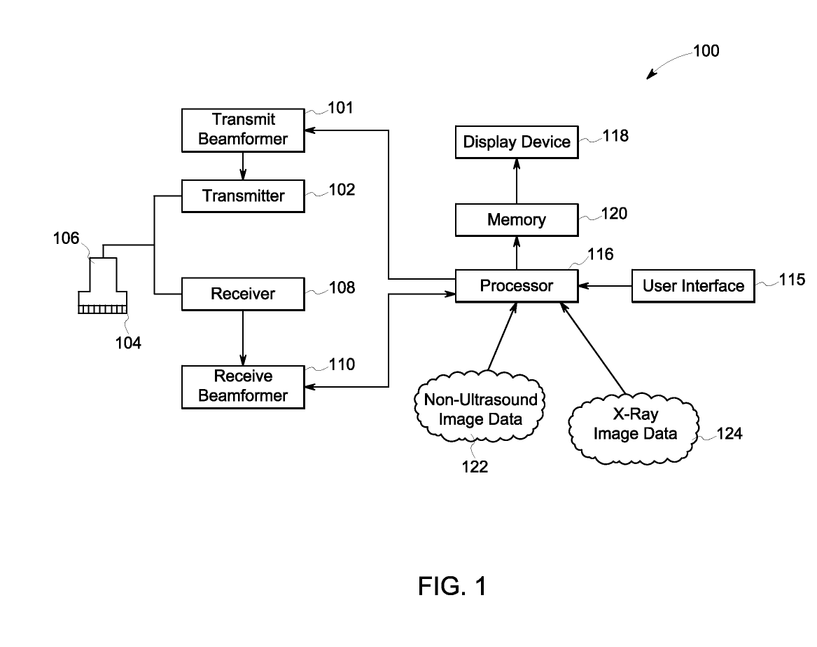

FIG. 1 is a schematic diagram of an ultrasound imaging system 100 in accordance with an embodiment. The ultrasound imaging system 100 includes a transmit beamformer 101 and a transmitter 102 that drive elements 104 within a probe 106 to emit pulsed ultrasonic signals into a body (not shown). The probe 106 may be any type of probe, including a linear probe, a curved array probe, a 1.25D array, a 1.5D array, a 1.75D array, or 2D array probe according to various embodiments. The probe 106 may be used to acquire 4D ultrasound data that contains information about how a volume changes over time. Each of the volumes may include a plurality of 2D images or slices. Still referring to FIG. 1, the pulsed ultrasonic signals are back-scattered from structures in the body, like blood cells or muscular tissue, to produce echoes that return to the elements 104. The echoes are converted into electrical signals, or ultrasound data, by the elements 104 and the electrical signals are received by a receiver 108. The electrical signals representing the received echoes are passed through a receive beamformer 110 that outputs ultrasound data. According to some embodiments, the probe 106 may contain electronic circuitry to do all or part of the transmit beamforming and/or the receive beamforming. For example, all or part of the transmit beamformer 101, the transmitter 102, the receiver 108 and the receive beamformer 110 may be situated within the probe 106. The terms “scan” or “scanning” may also be used in this disclosure to refer to acquiring data through the process of transmitting and receiving ultrasonic signals. The terms “data” and “ultrasound data” may be used in this disclosure to refer to either one or more datasets acquired with an ultrasound imaging system. A user interface 115 may be used to control operation of the ultrasound imaging system 100. The user interface may be used to control the input of patient data, or to select various modes, operations, and parameters, and the like. The user interface 115 may include a one or more user input devices such as a keyboard, hard keys, a touch pad, a touch screen, a track ball, rotary controls, sliders, soft keys, or any other user input devices.

The ultrasound imaging system 100 also includes a processor 116 to control the transmit beamformer 101, the transmitter 102, the receiver 108 and the receive beamformer 110. The receive beamformer 110 may be either a conventional hardware beamformer or a software beamformer according to various embodiments. If the receive beamformer 110 is a software beamformer, it may comprise one or more of the following components: a graphics processing unit (GPU), a microprocessor, a central processing unit (CPU), a digital signal processor (DSP), or any other type of processor capable of performing logical operations. The beamformer 110 may be configured to perform conventional beamforming techniques as well as techniques such as retrospective transmit beamforming (RTB).

The processor 116 is in electronic communication with the probe 106. The processor 116 may control the probe 106 to acquire ultrasound data. The processor 116 controls which of the elements 104 are active and the shape of a beam emitted from the probe 106. The processor 116 is also in electronic communication with a display device 118, and the processor 116 may process the ultrasound data into images for display on the display device 118. For purposes of this disclosure, the term “electronic communication” may be defined to include both wired and wireless connections. The processor 116 may include a central processing unit (CPU) according to an embodiment. According to other embodiments, the processor 116 may include other electronic components capable of carrying out processing functions, such as a digital signal processor, a field-programmable gate array (FPGA), a graphics processing unit (GPU) or any other type of processor. According to other embodiments, the processor 116 may include multiple electronic components capable of carrying out processing functions. For example, the processor 116 may include two or more electronic components selected from a list of electronic components including: a central processing unit (CPU), a digital signal processor (DSP), a field-programmable gate array (FPGA), and a graphics processing unit (GPU). According to another embodiment, the processor 116 may also include a complex demodulator (not shown) that demodulates the RF data and generates raw data. In another embodiment the demodulation can be carried out earlier in the processing chain. The processor 116 may be adapted to perform one or more processing operations according to a plurality of selectable ultrasound modalities on the data. The data may be processed in real-time during a scanning session as the echo signals are received. For the purposes of this disclosure, the term “real-time” is defined to include a procedure that is performed without any intentional delay. Real-time frame or volume rates may vary based on the size of the region or volume from which data is acquired and the specific parameters used during the acquisition. The data may be stored temporarily in a buffer (not shown) during a scanning session and processed in less than real-time in a live or off-line operation. Some embodiments of the invention may include multiple processors (not shown) to handle the processing tasks. For example, a first processor may be utilized to demodulate and decimate the RF signal while a second processor may be used to further process the data prior to display as an image. It should be appreciated that other embodiments may use a different arrangement of processors. For embodiments where the receive beamformer 110 is a software beamformer, the processing functions attributed to the processor 116 and the software beamformer hereinabove may be performed by a single processor such as the receive beamformer 110 or the processor 116. Or, the processing functions attributed to the processor 116 and the software beamformer may be allocated in a different manner between any number of separate processing components.

According to an embodiment, the ultrasound imaging system 100 may continuously acquire ultrasound data at a frame-rate of, for example, 10 Hz to 30 Hz. Images generated from the data may be refreshed at a similar frame-rate. Other embodiments may acquire and display data at different rates. For example, some embodiments may acquire ultrasound data at a frame rate of less than 10 Hz or greater than 30 Hz depending on the size of the volume and the intended application. A memory 120 is included for storing processed frames of acquired data. In an exemplary embodiment, the memory 120 is of sufficient capacity to store frames of ultrasound data acquired over a period of time at least several seconds in length. The frames of data are stored in a manner to facilitate retrieval thereof according to its order or time of acquisition. The memory 120 may comprise any known data storage medium.

Optionally, embodiments of the present invention may be implemented utilizing contrast agents. Contrast imaging generates enhanced images of anatomical structures and blood flow in a body when using ultrasound contrast agents including microbubbles. After acquiring data while using a contrast agent, the image analysis includes separating harmonic and linear components, enhancing the harmonic component and generating an ultrasound image by utilizing the enhanced harmonic component. Separation of harmonic components from the received signals is performed using suitable filters. The use of contrast agents for ultrasound imaging is well-known by those skilled in the art and will therefore not be described in further detail.

In various embodiments of the present invention, data may be processed by other or different mode-related modules by the processor 116 (e.g., B-mode, Color Doppler, M-mode, Color M-mode, spectral Doppler, Elastography, TVI, strain, strain rate, and the like) to form 2D or 3D images or data. For example, one or more modules may generate B-mode, color Doppler, M-mode, color M-mode, spectral Doppler, Elastography, TVI, strain, strain rate and combinations thereof, and the like. The image beams and/or frames are stored and timing information indicating a time at which the data was acquired in memory may be recorded. The modules may include, for example, a scan conversion module to perform scan conversion operations to convert the image frames from coordinates beam space to display space coordinates. A video processor module may be provided that reads the image frames from a memory and displays the image frames in real time while a procedure is being carried out on a patient. A video processor module may store the image frames in an image memory, from which the images are read and displayed.

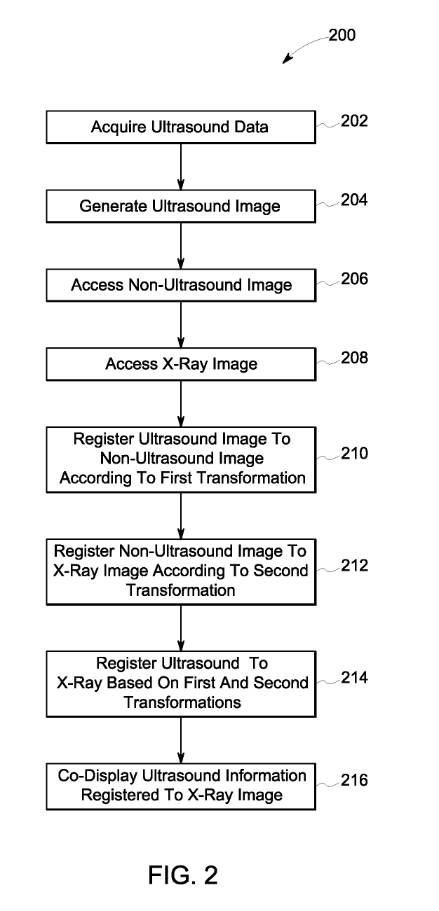

FIG. 2 is a flow chart of a method in accordance with an exemplary embodiment. The individual blocks of the flow chart represent steps that may be performed in accordance with the method 200. Additional embodiments may perform the steps shown in a different sequence and/or additional embodiments may include additional steps not shown in FIG. 2. The technical effect of the method 200 is the co-displaying of ultrasound information registered to an x-ray image.

Referring to FIGS. 1 and 2, at step 202, the controller 116 controls the transmit beamformer 101, the transmitter 102, and the probe 106 to acquire ultrasound data. The ultrasound data may comprise 2D ultrasound data or 3D ultrasound data.

At step 204, the processor 116 controls the generation of an ultrasound image based on the ultrasound data. The processor 116 may generate the ultrasound image based on the beamformed data received from the receive beamformer 110. Or, according to embodiments where the receive beamformer 110 comprises a software beamformer, the processor 116 may instruct the software beamformer to generate a particular type of image. The software beamformer may apply the appropriate delays to the ultrasound data in order to generate one or more frames of ultrasound images based on the ultrasound data. The software beamformer may also apply retrospective transmit beamforming (RTB) techniques to the ultrasound data. In order to perform RTB, two or more samples need to be acquired at each location, each with a different focus. The software beamformer then applies a time offset to at least one of the two or more samples acquired at each location, allowing the samples to be combined in-phase. The software beamformer next combines the samples and generates an image. According to other embodiments, the processor 116 may function as the software beamformer and perform some or all of the processing operations that were described as being performed by the software beamformer hereinabove.

At step 206, the processor 116 accesses a non-ultrasound image, such as by accessing non-ultrasound image data 122. The non-ultrasound image data 122, may comprise a non-ultrasound image in a format that is ready for display, or the non-ultrasound image data 122 may requires additional processing by the processor 116 prior to display as the non-ultrasound image. At step 208, the processor 116 accesses an x-ray image, such as by accessing x-ray image data 124. According to an exemplary embodiment, the non-ultrasound image data may comprise an image from another imaging modality, such as computed tomography (CT), magnetic resonance imaging (MRI), positron emission tomography (PET), single photon emission computed tomography (SPECT), or any other imaging modality other than ultrasound. The processor 116 may access the non-ultrasound image data directly from a separate diagnostic imaging device, from a database or memory, such as a picture archiving and communication system (PACS) or from any other device. The processor 116 may access the non-ultrasound image data 122 through either a wired or a wireless transmission. The non-ultrasound image data may be 3D data. According to an exemplary embodiment, the non-ultrasound image may comprise a CT image, but it should be appreciated that the non-ultrasound image may be any other type of image other than an ultrasound image as well. The non-ultrasound image data may comprise preoperative data that is acquired before starting the method 200.

The x-ray image data may comprise an x-ray fluoroscopy image. The x-ray image may also comprise a non-fluoroscopy x-ray image such as a conventional 2D radiology image. The x-ray image data may be in a format that is ready for display as an x-ray image, or the x-ray image data may require additional processing prior to display as an x-ray image. At step 210, the processor 116 registers the ultrasound image to the non-ultrasound image, such as a CT image, according to a first transformation. The processor 116 may calculate the first transformation by implementing a correlation function, such as a least squares algorithm. The correlation function may be used to calculate the transformation that minimizes the difference between ultrasound image and the non-ultrasound image. The first transformation may be either a rigid or a deformable transformation. It should be appreciated that the non-ultrasound image may include images from other modalities according to other embodiments. The ultrasound image may be a 2D image or a 3D image, but the method 200 will be described according to an exemplary embodiment where the ultrasound image is a 3D image. For embodiments where the non-ultrasound image is a 3D image, such as a CT image, the processor 116 is able to register the ultrasound image to the non-ultrasound image based on structures present in both the ultrasound image and the non-ultrasound image. The processor 116 may also be able to register the ultrasound image to the non-ultrasound image based by implementing other types of correlation algorithms.

In one exemplary embodiment, the method 200 may be used during an interventional cardiac procedure, though it should be appreciated that the method 200 may be used to register images for any other type of procedure as well. According to an embodiment, the processor 116 may identify and segment a common structure in both the ultrasound image and the non-ultrasound image. The segmentation may be fully automatic, semi-automatic, or manual according to various embodiments. According to both the semi-automatic and the manual embodiments, a clinician may be required to identify one or more common points between the ultrasound image and the non-ultrasound image. According to the fully automatic embodiments, the processor 116 may perform the segmentation without requiring the clinician to identify any shapes or anatomical landmarks in either of the images. For clinical situations where the images include the heart, structures such as the aortic root, the aortic tube, valves, ventricles or atria may be identified with an image processing algorithm and segmented from the images. Models of various anatomical structures may be generated before implementing the method 200, and the processor 116 may identify portions of the ultrasound image and the non-ultrasound image that represent the best fit to the previously generated models of the anatomical structure. The models may comprise 2D or 3D representations of one or more anatomical structures. For example, the model may include a geometric solid or a mesh with a shape and dimensions defined by a priori information, such as previous imaging exams or clinical data. According to an embodiment where both the ultrasound and non-ultrasound image are 3D images, the processor 116 may fit a deformable mesh to various surfaces in both images. Each mesh may, for instance, include a grid of vertices where each vertex is fit to a point on a surface represented in the 3D image. The processor 116 may next use the mesh to identify regions with shapes and sizes that are consistent with a specific structure. The processor 116 may use a correlation function, such as least squares, or any other function adapted to determine the difference between the mesh and the specific structure. The processor 116 may identify the anatomical structure in each image by identifying the portions of the meshes based on the ultrasound image and the non-ultrasound image respectively that most strongly correlate with the a prior information about the shape of the structure. The method 200 is particularly advantageous when registering a 3D ultrasound image to a 3D non-ultrasound image, such as a CT image. Since both the ultrasound image and the non-ultrasound image are 3D images, three-dimensional structures in the ultrasound image and the non-ultrasound image will have a high degree of similarity in both images. As such, the registration of the ultrasound image to the non-ultrasound image may be performed very accurately with either minimal or zero clinician input. For most situations, the processor 116 may obtain a more accurate registration when registering two 3D images to each other compared to situations where a 3D image is registered to a 2D image. It is additionally usually possible to obtain a more accurate registration between two 3D images compared to the registration that is possible between two 2D images unless both of the 2D images were obtained with exactly the same acquisition geometry.

At step 212, the processor 116 registers the non-ultrasound image to the x-ray image according to a second transformation. The processor 116 may calculate the second transformation by minimizing the differences calculated with a correlation function such as least squares. The processor 116 may calculate the transformation needed to minimize the cost function indicating the differences between the non-ultrasound image and the x-ray image. The second transformation may be either a rigid or a non-rigid transformation. It is particularly advantageous when the non-ultrasound image is a CT image or another x-ray based image since both the x-ray image and the CT image are generated with x-rays. The CT image and the x-ray image will share strong similarities because both images were acquired with the X-rays. For example, the relative intensities in the CT and the x-ray image will usually be more strongly correlated than the relative intensities in an x-ray image and a non-x-ray image. The commonalities between the x-ray image and the CT image allow the processor 116 to register the images more accurately, more quickly, and with a higher level of confidence since the registration algorithm may include assumptions possible only when either registering two images acquired with x-rays or when registering two images that are likely to have a high degree of correlation. For example, the processor 116 may be able to register the non-ultrasound image to the x-ray image according to a rigid transformation or the processor 116 may only need to make very minor deformations in order to register the two images to each other.

Next, at step 214, the processor 116 registers the ultrasound image to the x-ray image based on both the first transformation and the second transformation that were previously calculated. As described hereinabove, the first transformation represents the transformation needed to register the ultrasound image to the non-ultrasound image. The second transformation represents the transformation needed to register the non-ultrasound image to the x-ray image. Since both the first transformation and the second transformation are relative to the non-ultrasound image, the processor 116 may calculate the relative transformations needed to register the ultrasound image, the non-ultrasound image, and the x-ray image to each other with respect to a common coordinate system. The processor 116 may, for instance, calculate the first transformation and the second transformation with respect to a coordinate system based on any one of the images (i.e. the ultrasound image, the non-ultrasound image, or the X-ray image). Or the processor 116 may calculate the transformations with respect to an arbitrary coordinate system. The processor 116 may derive the transformation needed to register the ultrasound image with the x-ray image based on the information in the first transformation and the second transformation.

According to an exemplary embodiment, the processor 116 may calculate both the first and second transformations with respect to a coordinate system of the non-ultrasound image. The processor 116 may then calculate a third transformation needed to directly register the ultrasound image to the x-ray image based on the first and second transformations since the first and second transformations were calculated with respect to the same coordinate system.

At step 216, the processor 116 co-displays ultrasound information registered to the x-ray image. The ultrasound information may include an ultrasound image or any other information or data based on or derived from the ultrasound data.

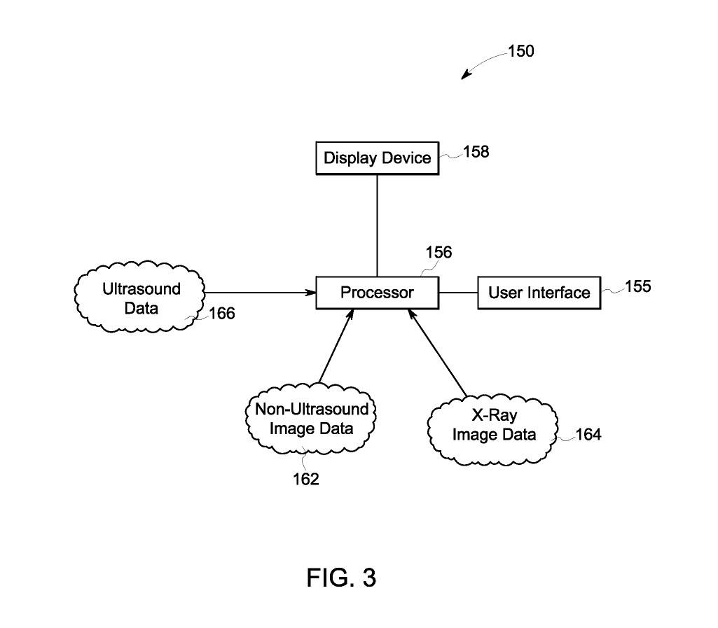

FIG. 3 is a schematic representation of a system 150 in accordance with an embodiment. The system 150 includes a user interface 155, a processor 156, and a display device 158. The user interface 155 may include a one or more user input devices such as a keyboard, hard keys, a touch pad, a touch screen, a track ball, rotary controls, sliders, soft keys, or any other user input devices. The user interface 155 may be configured to control or provide instructions to the processor 156. The processor 156 may, for instance, include one or more components selected from a central processing unit (CPU), a digital signal processor, a field-programmable gate array (FPGA), a graphics processing unit (GPU), or any other component capable of carrying out logical or processing functions. According to an embodiment, the system 150 may be configured to implement a modification of the previously described method 200. For example, steps 202 and 204 may be performed by an ultrasound imaging system that is separate from the system 150. The processor 156 may instead be configured to access ultrasound data 166 from either an ultrasound system or from a memory. The memory may, for example, be part of a picture archiving and communication system (PACS) or a database. According to an embodiment, the processor 156 may perform steps 206 and 208 where the processor 156 accesses non-ultrasound image data 162 and x-ray image data 164 respectively. The non-ultrasound image data 162 and the x-ray image data 164 may be accessed from the imaging systems used to acquire the image data, or they may be accessed from a PACS system or any other type of memory or database. The processor 156 may then implement steps 210, 212, 214, and 216. These steps were previously described according to an embodiment where there were performed with the processor 116 shown in FIG. 1 and, as such, they will not be described in detail with respect to the system 150. At step 216, the processor 156 co-displays ultrasound information that is registered to an x-ray image.

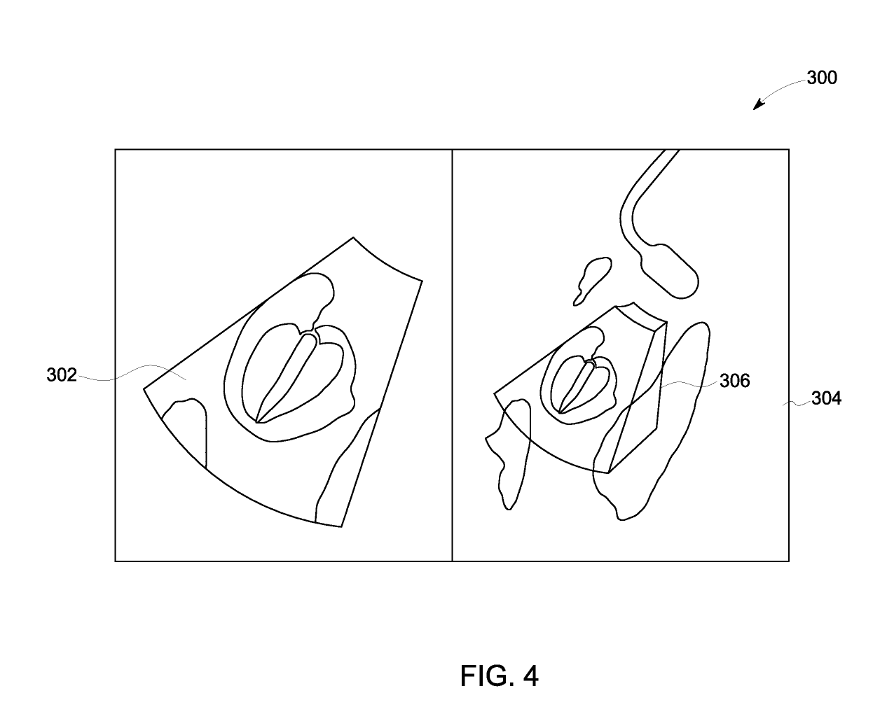

FIG. 4 is a schematic representation of a screen shot 300 according to an exemplary embodiment. The screenshot 300 may be displayed on a display device such as the display device 118. The screenshot 300 includes an ultrasound image 302 and an x-ray fluoroscopy image 304. The x-ray image 304 is a x-ray fluoroscopy image according to an embodiment. The ultrasound image 302 is registered to the x-ray fluoroscopy image 304. The ultrasound image 302 may comprise either a 2D image or a rendering of 3D ultrasound data, such as a volume-rendered image. Additionally, the ultrasound image 302 may comprise Doppler, colorflow, or other types of ultrasound data. The ultrasound image 302 is rotated so that it is in the same orientation as the x-ray image 304 according to an embodiment. It may be advantageous to display the x-ray image 304 and the ultrasound image 302 with a common relative orientation with respect to a structure to help the clinician remain oriented when changing focus between the x-ray image 304 and the ultrasound image 302 as shown in FIG. 3. Not all embodiments, however, may include displaying the ultrasound image 302 and the x-ray image 304 with a common relative orientation. In other embodiments, the ultrasound image 302 may be displayed in a standard orientation—i.e. with the portion of the image closest to the probe oriented to be at the top of the display device. However, the ultrasound image and the x-ray image may still be registered to each other so that the processor 116 may easily identify corresponding points in one image based on points identified in the other image.

Ultrasound information, such as outline 306, is co-displayed with the x-ray fluoroscopy image 304. The outline 306 represents the volume from which the ultrasound data was acquired. Other embodiments may include an outline showing a 2D region instead of a 3D volume from which the ultrasound data was acquired corresponding with 2D ultrasound modes. The ultrasound image 302 may be a live (real-time) ultrasound image. The ultrasound image 302 may update in real-time as additional ultrasound data is acquired. Any additional ultrasound information that is co-displayed with the x-ray image 304 may also be updated in real-time. For example, the outline 306 may be adjusted in real-time to accurately represent the most current acquisition region or volume. The embodiment depicted in FIG. 3 allows the user to obtain real-time feedback about the region or volume from which the ultrasound data is being acquired on the x-ray fluoroscopy image 304. By using the x-ray fluoroscopy image 304, the user is able to position a catheter or any other interventional device more clearly and accurately than would be possible with only an ultrasound image. Additionally, both the ultrasound information and the ultrasound image 302 may be updated in real-time without exposing the patient and/or the clinician to any additional ionizing radiation. This offers a significant benefit as the most prevalent conventional technique involves exposing both the patient and the clinician to ionizing radiation every time the clinician acquires additional x-ray fluoroscopy images. According to many conventional workflows, the x-ray fluoroscopy system may actively acquire x-ray data for all or at least a significant portion of the interventional procedure.

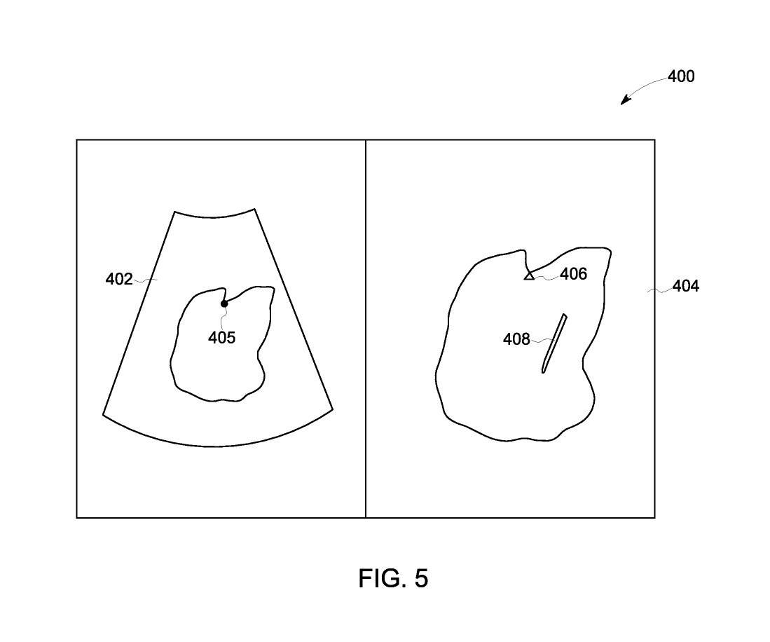

FIG. 5 is a schematic representation of a screenshot 400 in accordance with an embodiment. The screenshot 400 includes an ultrasound image 402, an x-ray image 404, and a marker 406. The ultrasound image 402 is registered to the x-ray image 404. Referring to the method 200 shown in FIG. 2, the ultrasound information comprises the marker 406 according an embodiment. According to an exemplary workflow, the clinician may identify a location 405 on the ultrasound image corresponding to a particular portion of the patient's anatomy, and then the processor 116 may display a marker, such as the marker 406 on the x-ray image 404. The marker 406 is positioned at a location corresponding to the location 405 identified by the clinician in the ultrasound image 402. The processor 116 may display one or more markers like the marker 406 on the x-ray image 404 as the clinician identifies various locations on the ultrasound image 402. Since the ultrasound image 402 is registered to the x-ray image 404, the processor 116 may quickly and accurately show any number of markers on the x-ray image corresponding to locations identified in the ultrasound image. The clinician is then able to easily discern the position of the markers with respect to an interventional device 408 that is clearly visible on the x-ray image. The clinician is therefore able to leverage the strengths of each imaging modality. For example, in some cardiac procedures, it is desirable to determine where leakage is occurring if any of the valves are not opening and closing properly. Ultrasound imaging modes, such as colorflow, are particularly well-suited for identifying areas with irregular flow. Additionally, ultrasound imaging is a particularly useful modality for imaging soft tissue. On the other hand, x-ray fluoroscopy is very well-suited for imaging the precise locations of interventional devices with respect to a patient's anatomy. According to an embodiment schematically shown in FIG. 5, the clinician is able to identify and mark the irregularities on the ultrasound image, and the processor 116 is then able to place one or more markers like marker 406 on the x-ray image 404. This allows the clinician to easily see the problematic areas with respect to the position of an interventional device on the x-ray image 404 that is registered to the ultrasound image 402.

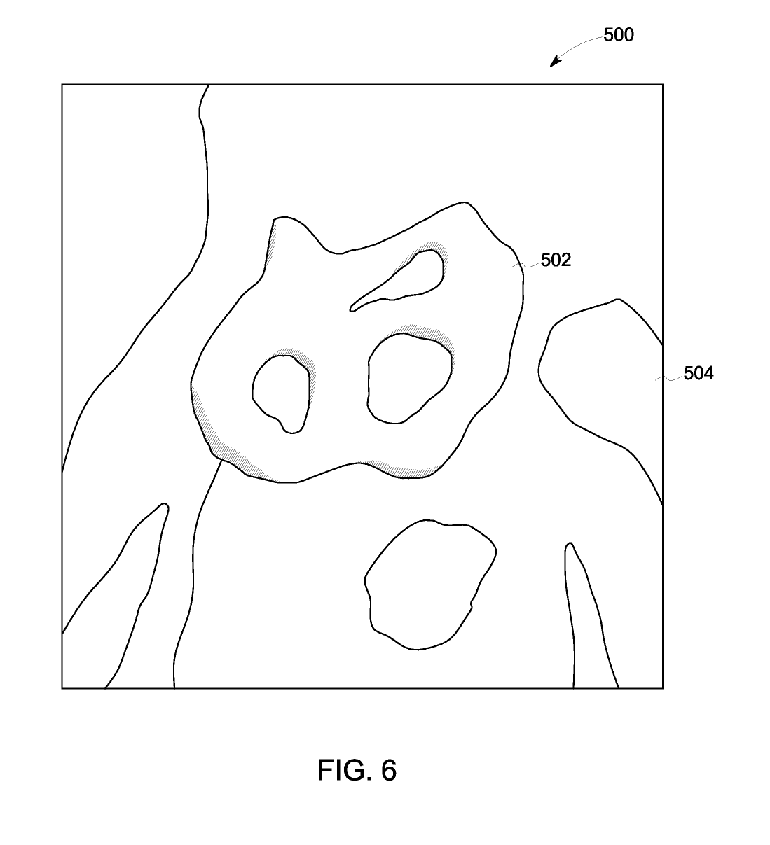

FIG. 6 is a schematic representation of a screenshot 500 in accordance with an embodiment. The screenshot 500 includes an ultrasound image 502 co-displayed with an x-ray image 504. The ultrasound image 502 is registered to the x-ray image 504 and the ultrasound image 502 is displayed as an overlay on top of the x-ray image. The ultrasound image 502 may comprise any ultrasound imaging mode, including B-mode, colorflow, or any other imaging mode. Additionally, the ultrasound image 502 may comprise a volume-rendered image. The volume-rendered image may likewise represent B-mode data, colorflow data, power Doppler data, or any other type of data acquired with an ultrasound imaging system. Additionally when displayed as an overlay, the ultrasound image 502 may include opaque lines and colorization. Or according to other embodiments, the ultrasound image 502 may comprise transparent or semi-transparent lines and colorization so that the user may see a portion of the underlying x-ray image 504 for reference. The ultrasound image 502 may also comprise a live ultrasound image registered to and co-displayed with the x-ray image so that the clinician may receive real-time information from the ultrasound image. The ultrasound image may be modified in real-time as additional ultrasound data is acquired. The processor 116 may repeat the registration of the newly acquired ultrasound data to the x-ray image 504 as additional ultrasound data are acquired. The processor 116 may repeat the registration each time an additional frame of data is acquired or the processor 116 may repeat the registration only after a predetermined number of frames have been acquired.

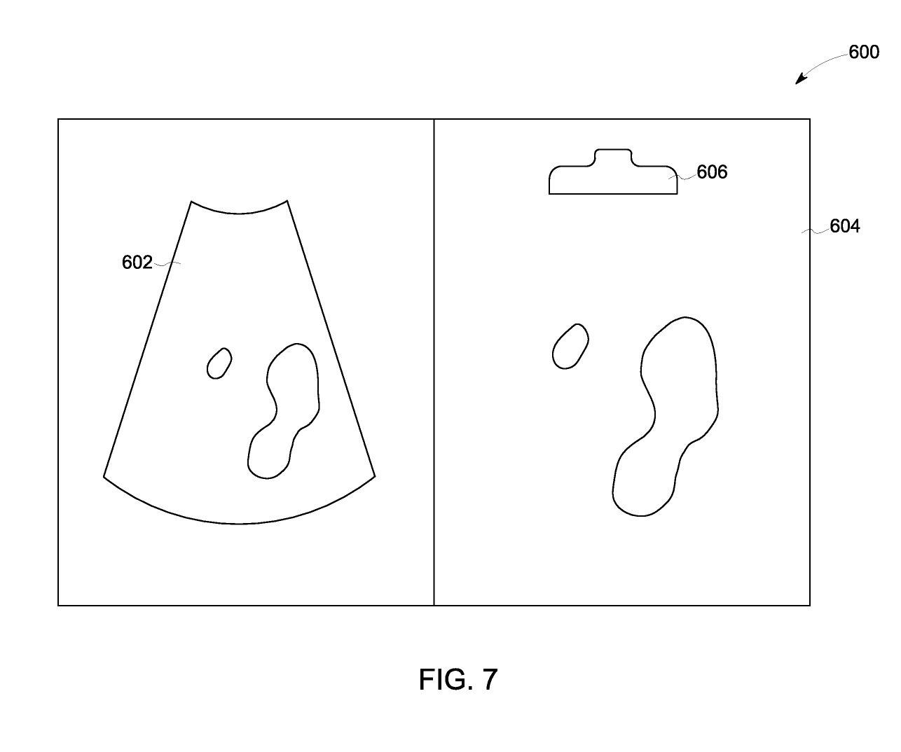

FIG. 7 is a schematic representation of a screenshot 600 according to an exemplary embodiment. The screenshot includes an ultrasound image 602, an x-ray image 604, and a graphic showing probe position 606. The graphic 606 is a schematic representation of the probe that may be co-displayed with the x-ray image 604 to help the clinician more easily understand the real-time position and orientation of the probe used to acquire the ultrasound data displayed in the ultrasound image 602. This is particularly helpful for cases where the orientation of the probe could be difficult to determine from only the x-ray image.

According to an embodiment, it may be desirable to detect if the probe 106 has moved while the x-ray image data is not being acquired. For example, according to an embodiment, the method 200 shown in FIG. 2 may be used to register a live or real-time ultrasound image to an x-ray image. If the probe 106 is moved from its initial position, the registration between the live ultrasound image and the x-ray image may no longer be accurate. As such, it may be desirable to have the processor 116 provide a warning or an indication to the user if the position of the probe 106 has moved from an original position of the probe 106. The warning or indication may include a text-based warning message displayed on the display device 118, an audible warning delivered through a speaker, a graphic displayed on the display device, or any other type of warning or message. The message, warning or indication may communicate to the user the need to acquire an updated x-ray image so that the registration between the x-ray image and the ultrasound image can be updated. Other embodiments may also provide warnings if any other parameters have changed that could render the registration between the x-ray image and the ultrasound image inaccurate.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method of medical imaging comprising:
accessing ultrasound data;
generating an ultrasound image based on the ultrasound data;
accessing a non-ultrasound image and an x-ray image;
registering the ultrasound image to the non-ultrasound image according to a first transformation;
registering the non-ultrasound image to the x-ray image according to a second transformation;
registering the ultrasound image to the x-ray image based on the first transformation and the second transformation; and
co-displaying ultrasound information registered to the x-ray image,
wherein the ultrasound information is based on the ultrasound data.

2. The method of claim 1, wherein the ultrasound image comprises a live ultrasound image.

3. The method of claim 2, wherein said registering the ultrasound image to the x-ray image is performed in real-time and, wherein said co-displaying the ultrasound information registered to the x-ray image is updated in real-time.

4. The method of claim 1, further comprising identifying a location on the ultrasound image, and wherein the ultrasound information comprises a marker indicating a corresponding location on the x-ray image.

5. The method of claim 1, wherein the ultrasound information comprises a graphic positioned on the x-ray image to indicate a region or volume from which the ultrasound data was acquired.

6. The method of claim 5, wherein the graphic comprises an outline of the region or the volume from which the ultrasound data was acquired.

7. The method of claim 6, wherein the probe is moved during the process of acquiring the ultrasound data, and wherein the graphic is adjusted in real-time to indicate the region or volume from which the ultrasound is being acquired.

8. The method of claim 1, wherein the ultrasound image and the non-ultrasound image both comprise 3D images.

9. The method of claim 8, wherein said registering the ultrasound image to the non-ultrasound image comprises implementing an image processing technique to identify a common structure in both the ultrasound image and the non-ultrasound image.

10. The method of claim 1, wherein the ultrasound information comprises the ultrasound image.

11. The method of claim 10, wherein said co-displaying the ultrasound information registered to the x-ray image comprises displaying the ultrasound image as an overlay on top of the x-ray image.

12. The method of claim 11, wherein the ultrasound image comprises a volume-rendered image.

13. The method of claim 1, wherein said co-displaying the ultrasound information registered to the x-ray image comprises displaying the x-ray image in a first portion of a display device and displaying the ultrasound image in a second portion of the display device, and wherein the x-ray image and the ultrasound image are both displayed with a common relative orientation with respect to a structure in both the x-ray image and the ultrasound image.

14. An ultrasound imaging system comprising:
a probe;
a display device; and
a processor in electronic communication with the probe and the display device, wherein the processor is configured to:
control the probe to acquire ultrasound data;
access a non-ultrasound image;
access an x-ray image;
calculate a first transformation to register the x-ray image to the non-ultrasound image;
calculate a second transformation to register the ultrasound image to the non-ultrasound image;
calculate a third transformation to register the ultrasound image to the x-ray image based on both the first transformation and the second transformation; and
co-display ultrasound information registered to the x-ray image on the display device, wherein the ultrasound information is based on the ultrasound data

15. The ultrasound imaging system of claim 14, wherein the processor is configured to update the ultrasound information registered to the x-ray image in real-time as additional ultrasound data is acquired.

16. The ultrasound imaging system of claim 14, wherein the ultrasound information includes a graphic showing at least one of a probe position and a position of a region or volume from which the ultrasound data was acquired.

17. The ultrasound imaging system of claim 14, wherein the processor is configured to update the graphic in real-time while an x-ray imaging system used to acquire the x-ray image is in an “OFF” state.

18. The ultrasound imaging system of claim 14, wherein the ultrasound information comprises a marker positioned on the x-ray image to indicate a structure identified based on the ultrasound image.

19. The ultrasound imaging system of claim 14, wherein the ultrasound image and the non-ultrasound image each comprise a 3D image, and wherein the processor is configured to identify and segment a common anatomical structure in both the ultrasound image and the non-ultrasound image.

20. The ultrasound imaging system of claim 19, wherein the processor is configured to segment the common anatomical structure in the ultrasound image and the non-ultrasound image by using an image processing technique involving a mesh.
